(19) United States
(12) Patent Application Publication
(10) Pub. No.: US 2019/0365316 A1
(43) Pub. Date: Dec. 5, 2019

(54) SENSORS FOR CARDIOTOXICITY MONITORING

(71) Applicant: Cardiac Pacemakers, Inc., St. Paul, MN (US)

(72) Inventors: Pramodsingh Hirasingh Thakur, Woodbury, MN (US); Viktoria A. Averina, Shoreview, MN (US); Gezheng Wen, Shoreview, MN (US); Jonathan Bennett Shute, Minnetonka, MN (US); Robert J. Sweeney, Woodbury, MN (US); Bin Mi, Arden Hills, MN (US)

(21) Appl. No.: 16/425,302

(22) Filed: May 29, 2019

Related U.S. Application Data
(60) Provisional application No. 62/678,085, filed on May 30, 2018.

(51) Int. Cl. A61B 5/00; A61B 5/029; A61B 5/024; A61B 5/0205; A61B 5/053; G16H 20/10

(52) U.S. Cl. CPC A61B 5/4839; A61B 5/0004; A61B 5/7271; A61B 5/029; A61B 5/746; A61B 5/0205; A61B 5/053; G16H 20/10; A61B 5/024

(57) ABSTRACT

This document discusses, among other things, systems and methods to receive physiologic information from a patient using an ambulatory medical device, and to determine an indication of cardiotoxicity using the received physiologic information.

### CLAIM OF PRIORITY

This application claims the benefit of priority under 35 U.S.C. § 119(e) of U.S. Provisional Patent Application Ser. No. 62/678,085, filed on May 30, 2018, which is herein incorporated by reference in its entirety.

### TECHNICAL FIELD

This document relates generally to medical devices, and more particularly, but not by way of limitation, to systems, devices, sensors, and methods for cardiotoxicity monitoring.

### BACKGROUND

Cancer is a major worldwide public health concern and the second leading cause of death in the United States. 1.7 million new cancer cases and over 600,000 cancer deaths are projected to occur in 2018. With early detection and improved therapy, cancer survivorship reached 14.5 million patients in 2014, and is projected to exceed 19 million by 2024.

Side effects of chemotherapy treatments vary from patient-to-patient, depending on the type of medication and length of treatment. For example, anthracyclines, cyclophosphamide, and trastuzumab may impact cardiac function (e.g., contractility). Other medications, such as imatinib, impact cardiac decompensation by altered preload (e.g., fluid retention). Bevacizumab may impact cardiac decompensation by altered afterload (e.g., hypertension). Ifosfamide may impact heart rate and arrhythmias. 5-cisplatin and 5-fluorouracil may impact cerebrovascular disease (e.g., ischemia risk), etc.

Cardiotoxicity is the occurrence of heart electrophysiology dysfunction or muscle damage resulting in a weak and inefficient cardiac supply, and is often a result of cancer treatment, affecting between 5% and 65% of cancer treatment patients. Cardiotoxicity is currently monitored by point-of-care diagnostics, and largely after cancer treatment.

### SUMMARY

This document discusses, among other things, systems and methods to receive physiologic information from a patient using an ambulatory medical device, and to determine an indication of cardiotoxicity using the received physiologic information.

An example (e.g., “Example 1”) of subject matter (e.g., a system) may include an ambulatory medical device configured to receive physiologic information from a patient; and an assessment circuit configured to determine an indication of cardiotoxicity using the received physiologic information.

In Example 2, the subject matter of Example 1 may optionally be configured to include a drug delivery system configured to control delivery of a cancer drug to the patient according to a delivery parameter, wherein the assessment circuit is configured to determine an optimized delivery parameter using the determined indication of cardiotoxicity.

In Example 3, the subject matter of any one or more of Examples 1-2 may optionally be configured such that the delivery parameter includes at least one of a dosage, a timing, or a drug.

In Example 4, the subject matter of any one or more of Examples 1-3 may optionally be configured such that the assessment circuit is configured to provide an alert to a user using the determined indication of cardiotoxicity.

In Example 5, the subject matter of any one or more of Examples 1-4 may optionally be configured such that the assessment circuit is configured to determine an adjusted therapy parameter using the determined indication of cardiotoxicity.

In Example 6, the subject matter of any one or more of Examples 1-5 may optionally be configured to include a therapy circuit configured to control a therapy to the patient according to a therapy parameter, wherein the assessment circuit is optionally configured to adjust the therapy parameter using the determined indication of cardiotoxicity.

In Example 7, the subject matter of any one or more of Examples 1-6 may optionally be configured such that the assessment circuit is configured to determine a cardiotoxicity index for the patient using the received physiologic information, to compare the determined cardiotoxicity index to a threshold, and, in response to the determined cardiotoxicity index exceeding the threshold, to adjust the therapy parameter using the determined cardiotoxicity index.

In Example 8, the subject matter of any one or more of Examples 1-7 may optionally be configured such that the ambulatory medical device includes a heart sound sensor configured to receive heart sound information of the patient, and the assessment circuit is configured to determine the indication of cardiotoxicity using the received heart sound information.

In Example 9, the subject matter of any one or more of Examples 1-8 may optionally be configured such that the assessment circuit is configured to determine an indication of reduced contractile function using the received heart sound information, and to determine the indication of cardiotoxicity using the determined indication of reduced contractile function.

In Example 10, the subject matter of any one or more of Examples 1-9 may optionally be configured such that the assessment circuit is configured to detect a decrease in first heart sound (S1) amplitude using the received heart sound information, to determine the indication of reduced contractile function using the detected decrease in first heart sound (S1) amplitude, and to determine the indication of cardiotoxicity using the determined indication of reduced contractile function.

An example (e.g., “Example 11”) of subject matter (e.g., at least one machine-readable medium) may include instructions that, when performed by a medical device, cause the medical device to receive physiologic information from a patient; and to determine an indication of cardiotoxicity using the received physiologic information.

In Example 12, the subject matter of Example 11 may optionally be configured to include instructions that, when performed by the medical device, cause the medical device to: control delivery of a cancer drug to the patient according to a delivery parameter; and determine an optimized delivery parameter using the determined indication of cardiotoxicity.

In Example 13, the subject matter of any one or more of Examples 11-12 may optionally be configured such that the delivery parameter includes at least one of a dosage, a timing, or a drug.

In Example 14, the subject matter of any one or more of Examples 11-13 may optionally be configured such that the instructions, when performed by the medical device, cause the medical device to: provide an alert to a user using the determined indication of cardiotoxicity.

In Example 15, the subject matter of any one or more of Examples 11-14 may optionally be configured such that the instructions, when performed by the medical device, cause the medical device to: determine an adjusted therapy parameter using the determined indication of cardiotoxicity.

An example (e.g., “Example 16”) of subject matter (e.g., a method) may include receiving physiologic information from a patient using an ambulatory medical device; and determining, using an assessment circuit, an indication of cardiotoxicity using the received physiologic information.

In Example 17, the subject matter of Example 16 may optionally be configured to include controlling, using the assessment circuit, delivery of a cancer drug to the patient according to a delivery parameter; and determining, using the assessment circuit, an optimized delivery parameter using the determined indication of cardiotoxicity.

In Example 18, the subject matter of any one or more of Examples 16-17 may optionally be configured such that the delivery parameter includes at least one of a dosage, a timing, or a drug.

In Example 19, the subject matter of any one or more of Examples 16-18 may optionally be configured to include providing, using the assessment circuit, an alert to a user using the determined indication of cardiotoxicity.

In Example 20, the subject matter of any one or more of Examples 16-19 may optionally be configured to include determining, using the assessment circuit, an adjusted therapy parameter using the determined indication of cardiotoxicity.

An example (e.g., “Example 21”) of subject matter (e.g., a system or apparatus) may optionally combine any portion or combination of any portion of any one or more of Examples 1-20 to include “means for” performing any portion of any one or more of the functions or methods of Examples 1-20, or a “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example relationship of systolic blood pressure measurements at different pacing patterns.

FIG. 2 illustrates an example system including an ambulatory medical device (AMD) configured to sense or detect information from a patient.

FIG. 3 illustrates an example system (e.g., a medical device, etc.) including a signal receiver circuit and an assessment circuit.

FIG. 4 illustrates an example system including an ambulatory medical device (AMD) coupled to an external or remote system, such as an external programmer.

FIG. 5 illustrates an example of a Cardiac Rhythm Management (CRM) system and portions of an environment in which the CRM system can operate.

FIG. 6 illustrates a block diagram of an example machine upon which any one or more of the techniques (e.g., methodologies) discussed herein may perform.

### DETAILED DESCRIPTION

The present inventor has recognized, among other things, that continuous monitoring, such as through an ambulatory medical device, including a wearable or implantable sensor, can enable early detection and better management of cardiotoxicity in cancer patients, potentially significantly expanding insertable cardiac monitor (ICM) or other ambulatory medical device indications.

Cardiotoxicity is a continuum, ranging from reversible to irreversible. Early recognition of cardiotoxicity is critical for treatment. Traditional early indications of sub-clinical myocardial injury were obtained through invasive blood draw (e.g., blood biomarkers, such as Troponin) or strain imaging, at a substantial cost or patient burden. If detected early, cardiotoxicity can be managed or reversed, such as using cardioprotective drugs (e.g., beta-blockers or ace-inhibitors, etc.). Later indications of cardiotoxicity can include echo-based left ventricular ejection fraction (LVEF) assessment. However, such measurements provide a single, intermittent assessment (e.g., every 6 months) in a clinical setting, which is undesirable for cancer patients in an immunocompromised state. If cardiotoxicity progresses without detection or treatment, it can become irreversible, leading to patient death.

TABLE 1
Example chemotherapeutics and possible cardiovascular damage

| Example Chemotherapeutics | Possible Cardiovascular Damage |
|---|---|
| Anthracyclines and | CHF, LVD, Acute Myocarditis, |
| Anthraquinolones | Arrhythmia |
| Capecitabine, 5-fluorouracil, | Ischemia, Pericarditis, CHF, |
| Cytarabine | Cardiogenic Shock |
| Paclitaxel, Vinca Alkaloids | Sinus Bradycardia, Ventricular |
| | Tachycardia, Atrioventricular Block, |
| | Hypotension, CHF, Ischemia |
| Cyclophosphamide | Neurohumoral Activation, Mitral |
| | Regurgitation |
| Imatinib | Arrhythmias, CHF, Angioedema, LVD |
| Sorafenib | Hypertension, Arrhythmias |
| Sunitinib | Hypertension, Arrhythmias |
| Selective Estrogen Receptor | LDL/HDL modulation, |
| Mudulators (SERMs) | Thromboembolism |
| Trastuzumab | Arrhythmias, CHF, Angioedema, LVD |
| Bevacizumab | Hypertension, Thromboembolism, GI |
| | Tract Bleeding |
| COX-2-specific Inhibitors | Thromboembolism |
| Thorax Irradiation | Myocardial Fibrosis, Valvular Heart |
| | Disease, LVD |

In an example, one or more existing ambulatory medical device sensors can be used to detect early subclinical manifestations of cardiotoxicity, during or after cancer treatment to a patient, such as to pre-screen for further medical intervention or therapy optimization (e.g., drug titration or timing, etc.). Such advancements can provide for early detection of treatable conditions, in certain examples providing additional use for existing sensors, reducing sensor cost, and enabling earlier intervention, improving patient outcomes, and reducing overall medical system costs. The systems and methods described herein, in certain examples, represent an improved form of cardiotoxicity detection and patient intervention over existing techniques. In certain examples, patients can be monitored, and the patient, caregiver, clinician, or one or more other system or user can be alerted to a change in patient condition indicative of cardiotoxicity, or a likelihood of cardiotoxicity. In other examples, the systems and methods described herein can provide a recommended intervention or therapy optimization (e.g., dosage or timing change, change in prescribed drug, etc.), or can directly provide or alter a therapy to the patient.

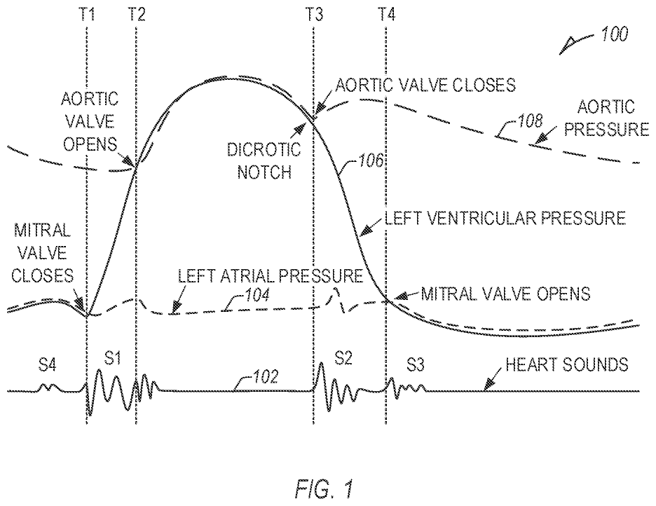

FIG. 1 illustrates an example relationship 100 between heart sounds 102, including first, second, third, and fourth heart sounds (S1, S2, S3, and S4), left atrial pressure 104, left ventricular pressure 106, and aortic pressure 108.

At a first time (T1), a mitral valve closes, marking a rise in left ventricular pressure 106, and the start of the first heart sound (S1) and systole, or ventricular contraction. At a second time (T2), an aortic valve opens, marking a rise in aortic pressure 108 and continuing S1. S1 is caused by closure of the atrioventricular (AV) valves, including the mitral and tricuspid valves, and can be used to monitor heart contractility.

At a third time (T3), an aortic valve closes, causing a dicrotic notch in the aortic pressure 108 and the second heart sound (S2), and marking the end of systole, or ventricular contraction, and the beginning of diastole, or ventricular relaxation. S2 can be used to monitor blood pressure. At a fourth time (T4), the mitral valve opens, and the left atrial pressure 104 drops. An abrupt halt of early diastolic filling can cause the third heart sound (S3), which can be indicative of (or an early sign of) heart failure (HF). Vibrations due to atrial kick can cause the fourth heart sound (S4), which can be used to monitor ventricular compliance.

Systolic time intervals, such as pre-ejection period (PEP) or left ventricular ejection time (LVET) can be indicative of clinically relevant information, including contractility, arrhythmia, Q-T prolongation (with electrogram (EGM) information), etc. The PEP can be measured from a Q wave of an EGM to the time of the aortic valve opening, T2 in FIG. 1. The LVET can include a time between the aortic valve opening, T2, and the aortic valve closing, T3. In other examples, one or more systolic time intervals can be detected and used to detect physiologic information of a patient (e.g., PEP/LVET, one or more mechanical, electrical, or mechanical-electrical time intervals, etc.).

Ambulatory medical devices, including implantable, leadless, or wearable medical devices configured to monitor, detect, or treat various cardiac conditions associated with a reduced ability of a heart to sufficiently deliver blood to a body, such as heart failure (HF), arrhythmias, hypertension, etc. Various ambulatory medical devices can be implanted in a patient's body or otherwise positioned on or about the patient to monitor patient physiologic information, such as heart sounds, respiration (e.g., respiration rate, tidal volume, etc.), impedance (e.g., thoracic impedance), pressure, cardiac activity (e.g., heart rate (HR)), physical activity, posture, or one or more other physiologic parameters of a patient, or to provide electrical stimulation or one or more other therapies or treatments to optimize or control contractions of the heart.

Traditional cardiac rhythm management (CRM) devices, such as pacemakers, defibrillators, or cardiac monitors, include implanted devices (e.g., implantable cardioverter-defibrillators (ICDs), etc.), subcutaneous devices (e.g., subcutaneous ICDs (S-ICDs), etc.), or one or more other devices configured to be implanted within in a chest of a patient, or under the skin of the patient, in certain examples, having one or more leads to position one or more electrodes or other sensors at various locations in the heart, such as in one or more of the atria or ventricles. Separate from, or in addition to, the one or more electrodes or other sensors of the leads, the CRM device can include one or more electrodes or other sensors (e.g., a pressure sensor, an accelerometer, a gyroscope, a microphone, etc.) powered by a power source in the CRM device. The one or more electrodes or other sensors of the leads, the CRM device, or a combination thereof, can be configured detect physiologic information from, or provide one or more therapies or stimulation to, the patient, for example, using one or more stimulation circuits.

Leadless cardiac pacemakers (LCP) include small (e.g., smaller than traditional implantable CRM devices), self-contained devices configured to detect physiologic information from or provide one or more therapies or stimulation to the heart without traditional lead or implantable CRM device complications (e.g., required incision and pocket, complications associated with lead placement, breakage, or migration, etc.). In certain examples, an LCP can have more limited power and processing capabilities than a traditional CRM device; however, multiple LCP devices can be implanted in or about the heart to detect physiologic information from, or provide one or more therapies or stimulation to, one or more chambers of the heart. The multiple LCP devices can communicate between themselves, or one or more other implanted or external devices.

Wearable or external medical sensors or devices can be configured to detect or monitor physiologic information of the patient without required implant or an in-patient procedure for placement, battery replacement, or repair. However, such sensors and devices, in contrast to implantable, subcutaneous, or leadless medical devices, may have reduced patient compliance, increased detection noise, or reduced detection sensitivity.

Determination of one or more patient conditions (e.g., hypertension, HF, etc.), or risk stratification for one or more patient conditions, often requires some initial assessment time to establish a baseline level or condition from one or more sensors or physiologic information from which a detected deviation is indicative of the patient condition, or risk of patient condition or future adverse medical event (e.g., the risk of the patient experiencing a heart failure event (HFE) within a following period, etc.). Changes in physiologic information can be aggregated and weighted based on one or more patient-specific stratifiers. However, such changes and risk stratification are often associated with one or more thresholds, for example, having a clinical sensitivity and specificity across a target population with respect to a specific condition (e.g., HF), etc., and one or more specific time periods, such as daily values, short-term averages (e.g., daily values aggregated over a number of days), long-term averages (e.g., daily values aggregated over a number of short-term periods or a greater number of days (sometimes different days than used for the short-term average)), etc.

For example, a multisensor algorithm has been demonstrated to predict HF events in patients with a high sensitivity and low false positive rate using physiologic information detected from one or more implanted or ambulatory medical devices. In other examples, such algorithm can be applied to one or more other medical events, such as hypertension or one or more conditions associated with hypertension, etc. The multisensor algorithm can determine a composite physiologic parameter using one or more of the following physiologic information: heart sounds (e.g., a first heart sound (S1), a second heart sound (S2), a third heart sound (S3), a fourth heart sound (S4), heart-sounds related time intervals, etc.), thoracic impedance (TI), respiratory rate (RR), rapid shallow breathing index (RSBI), heart rate (HR) (e.g., nighttime HR), activity, posture, cardiac activity, pressure, etc.

In certain examples, such multisensor algorithm can be adjusted using a determined patient risk level (e.g., a stratifier). The combination of or weight of respective primary and secondary sensors used to determine the composite physiologic parameter can be adjusted using the determined patient risk level. For example, if the determined patient risk level indicates a low risk of a worsening physiologic condition, the composite physiologic parameter can be determined using one or more primary sensors (and not one or more secondary sensors). If the determined patient risk level indicates a medium or high risk of worsening heart failure, the composite physiologic parameter can be determined using the primary sensors and a combination of the secondary sensors, depending on the determined patient risk level. In an example, the determined patient risk level or the determined risk of worsening heart failure can be used to determine an indication of cardiotoxicity, and to provide an alert, a recommended intervention or change in parameter or therapy, or to directly change or provide a therapy to the patient.

In an example, S1 amplitude can be a marker of contractile function (e.g., a decrease in S1 amplitude, or a decrease in the change of S1 amplitude, can be indicative of reduced contractility or contractile function, and vice versa, etc.). Systolic time intervals (e.g., PEP, PEP/LVET, etc.) can be indicative of contractile function (e.g., an increase in PEP or PEP/LVET can be indicative of a decrease in contractility). Low contractility, or a decrease in contractility, can be indicative of a decrease in cardiac function, and accordingly, in combination with cancer treatment, an increased likelihood of cardiotoxicity. Accordingly, S1 or systolic time intervals indicative of cardiac contractility can be used as an early indicator of cardiotoxicity.

In an example, S2 amplitude can be a marker of afterload changes (e.g., an increase in S2 can be indicative of increased afterload, and a reduction of stroke volume, etc.) As afterload increases, cardiac output decreases. A decrease in stroke volume, or cardiac output, can be indicative of a decrease in cardiac function, and accordingly, in combination with cancer treatment, an increased likelihood of cardiotoxicity. Accordingly, S2 can be indicative of afterload, stroke volume, or cardiac output, and can be used as an early indicator of cardiotoxicity.

In an example, S3 amplitude or impedance can be used to track fluid or preload changes, and further, can be an early indicator of worsening heart failure (HF). An increase in S3 (or S4) can be indicative of decreased cardiac output. Further, changes in respiratory rate (e.g., median respiratory rate trend (RRT) (minimally impacted by activity or exercise), etc.), tidal volume, heart rate (e.g., resting heart rate), or combinations thereof, can be indicative of decreased cardiac output (e.g., an increased respiratory rate, tidal volume, or heart rate (e.g., resting heart rate) can be indicative of decreased cardiac output) and can accordingly be used as an early indicator of cardiotoxicity.

In an example, one or more electrical, mechanical, or electrical-mechanical intervals can be used to track cardiac output or one or more other conditions, such as Q-T prolongation, etc., indicative of a decrease in cardiac output. For example, one or more of a Q-T interval, an R-T interval, an R-S2 interval, or one or more other electrical, mechanical, or electrical-mechanical intervals can be indicative of Q-T prolongation (e.g., an increase in one or more of the Q-T, R-T, or R-S2 interval can be indicative of an increase in Q-T prolongation, which can be indicative of arrhythmia, or a decrease in cardiac output, etc.), which, in combination with cancer treatment, can be indicative of an increased likelihood of cardiotoxicity.

In an example, one or more ambulatory medical devices can be configured to monitor patient response to a stimulus, such as a prescreening dose of water or saline, to detect patient response. Patients responding abnormally to such stimulus (e.g., water or saline) can be screened for further intervention or continued monitoring. In other examples, separate mechanisms (cause/effect) can be monitored by comparing temporal evolution of sensors (e.g., comparing a rate of S1 change versus intrathoracic impedance (ITTI), etc.), and the temporal evolution of the sensors can be indicative of an increased likelihood of cardiotoxicity.

In an example, the systems and methods described herein can be used to adjust or optimize cancer treatment to the patient. In certain examples, cancer treatments are configured to harm the patient to kill cancerous cells or stop or slow the growth of cancer cells at a moderate to severe impact to patient health, such as worsening heart failure or a risk of worsening heart failure. Higher dosages of cancer drugs can be better for cancer treatment, but worse for the patient's heart. During cancer treatment, adverse cardiac impact may be less weighty than the efficacy of cancer treatment, but only to a point. Currently, cardiac monitoring is not part of cancer treatment. The systems and methods described herein can be used to monitor indications of cardiac output, during or after cancer treatment, to optimize treatment (e.g., infusion, dosage, timing, etc.), in certain examples, increasing the harm to patients up to a desired level, to optimize cancer treatment without fatally harming the patient (e.g., up to a desired or determined “safety” level, etc.). Similarly, following cancer treatment, the systems and methods described herein can be used to optimize one or more recovery treatments or therapies.

In an example, if a patient has an existing cardiac monitor or ambulatory medical device, the existing devices may switch modes to implement the systems and methods described herein. In other examples, one or more additional ambulatory medical devices can be deployed to perform the systems and methods described herein.

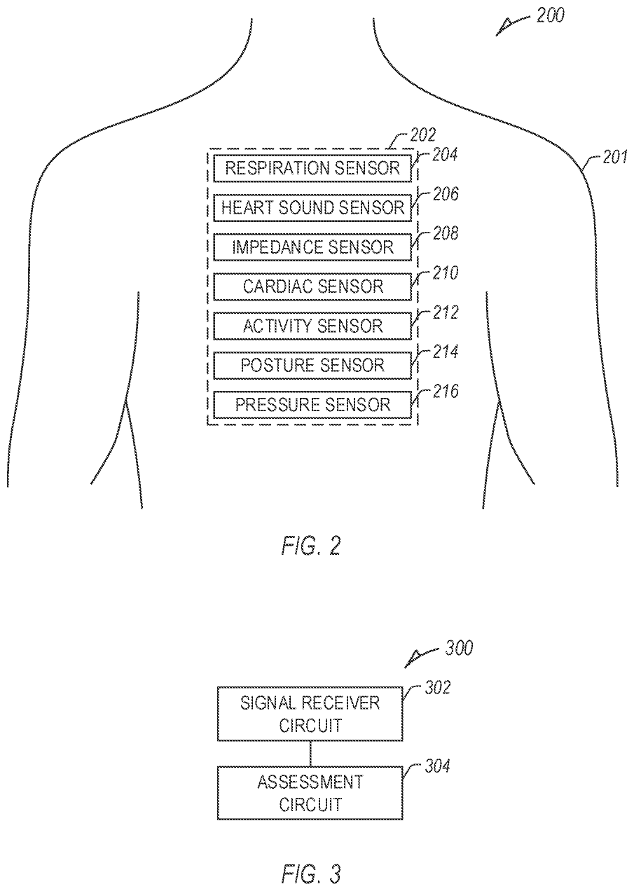

FIG. 2 illustrates an example system 200 including an ambulatory medical device (AMD) 202 configured to sense or detect information from a patient 201. In an example, the AMD 202 can include an implantable medical device (IMD), a subcutaneous or leadless medical device, a wearable or external medical device, or one or more other implantable or external medical devices or patient monitors. The AMD 202 can include a single device, or a plurality of medical devices or monitors configured to detect patient information.

The AMD 202 can include one or more sensors configured to receive physiologic information of a patient 201. In an example, the AMD 202 can include one or more of a respiration sensor 204 configured to receive respiration information (e.g., a respiration rate (RR), a respiration volume (tidal volume), etc.), a heart sound sensor 206 configured to receive heart sound information, an impedance sensor 208 (e.g., intrathoracic impedance sensor, transthoracic impedance sensor, etc.) configured to receive impedance information, a cardiac sensor 210 configured to receive cardiac electrical information, an activity sensor 212 configured to receive information about a physical motion (e.g., activity, steps, etc.), a posture sensor 214 configured to receive posture or position information, a pressure sensor 216 configured to receive pressure information, or one or more other sensors configured to receive physiologic information of the patient 201.

FIG. 3 illustrates an example system (e.g., a medical device, etc.) 300 including a signal receiver circuit 302 and an assessment circuit 304. The signal receiver circuit 302 can be configured to receive patient information, such as physiologic information of a patient (or group of patients) from one or more sensors. The assessment circuit 304 can be configured to receive information from the signal receiver circuit 302, and to determine one or more parameters (e.g., composite physiologic parameters, stratifiers, one or more pacing parameters, etc.), such as described herein.

The assessment circuit 304 can be configured to provide an output to a user, such as to a display or one or more other user interface, the output including a score, a trend, or other indication. In other examples, the assessment circuit 304 can be configured to provide an output to another circuit, machine, or process, such as to control, adjust, or cease a therapy of a medical device, a drug delivery system, etc.

FIG. 4 illustrates an example system 400 including an ambulatory medical device (AMD) 402 coupled to an external or remote system 404, such as an external programmer, and a drug delivery device 406, such as a device configured to deliver one or more drugs (e.g., cancer drugs) to a patient. In an example, the AMD 402 can be an implantable device, an external device, or a combination or permutation of one or more implantable or external devices. In an example, one or more of the signal receiver circuit 302 or the assessment circuit 304 can be located in the AMD 402, or the remote system 404. In an example, the AMD 402 can include a stimulation circuit configured to generate one or more pacing or defibrillation waveforms to be provided to a patient. The remote system 404 can include a specialized device configured to interact with the AMD 402, including to program or receive information from the AMD 402. The drug delivery device 406 can be configured to send information to or receive information from one or both of the AMD 402 or the remote system 404. In an example, the AMD 402 or the remote system 404 can be configured to control one or more parameters of the drug delivery system 406.

FIG. 5 illustrates an example of a Cardiac Rhythm Management (CRM) system 500 and portions of an environment in which the CRM system 500 can operate. The CRM system 500 can include an ambulatory medical device, such as an implantable medical device (IMD) 510 that can be electrically coupled to a heart 505 such as through one or more leads 508A-C coupled to the IMD 510 using a header 511, and an external system 520 that can communicate with the IMD 510 such as via a communication link 503. The IMD 510 may include an implantable cardiac device such as a pacemaker, an implantable cardioverter-defibrillator (ICD), or a cardiac resynchronization therapy defibrillator (CRT-D). The IMD 510 can include one or more monitoring or therapeutic devices such as a subcutaneously implanted device, a wearable external device, a neural stimulator, a drug delivery device, a biological therapy device, or one or more other ambulatory medical devices. The IMD 510 may be coupled to, or may be substituted by a monitoring medical device such as a bedside or other external monitor.

The IMD 510 can include a hermetically sealed can 512 that can house an electronic circuit that can sense a physiologic signal in the heart 505 and can deliver one or more therapeutic electrical pulses to a target region, such as in the heart, such as through one or more leads 508A-C. In certain examples, the CRM system 500 can include only a single lead, such as 508B, or can include only two leads, such as 508A and 508B.

The lead 508A can include a proximal end that can be configured to be connected to IMD 510 and a distal end that can be configured to be placed at a target location such as in the right atrium (RA) 531 of the heart 505. The lead 508A can have a first pacing-sensing electrode 551 that can be located at or near its distal end, and a second pacing-sensing electrode 552 that can be located at or near the electrode 551. The electrodes 551 and 552 can be electrically connected to the IMD 510 such as via separate conductors in the lead 508A, such as to allow for sensing of the right atrial activity and optional delivery of atrial pacing pulses. The lead 508B can be a defibrillation lead that can include a proximal end that can be connected to IMD 510 and a distal end that can be placed at a target location such as in the right ventricle (RV) 532 of heart 505. The lead 508B can have a first pacing-sensing electrode 552 that can be located at distal end, a second pacing-sensing electrode 553 that can be located near the electrode 552, a first defibrillation coil electrode 554 that can be located near the electrode 553, and a second defibrillation coil electrode 555 that can be located at a distance from the distal end such as for superior vena cava (SVC) placement. The electrodes 552 through 555 can be electrically connected to the IMD 510 such as via separate conductors in the lead 508B. The electrodes 552 and 553 can allow for sensing of a ventricular electrogram and can optionally allow delivery of one or more ventricular pacing pulses, and electrodes 554 and 555 can allow for delivery of one or more ventricular cardioversion/defibrillation pulses. In an example, the lead 508B can include only three electrodes 552, 554 and 555. The electrodes 552 and 554 can be used for sensing or delivery of one or more ventricular pacing pulses, and the electrodes 554 and 555 can be used for delivery of one or more ventricular cardioversion or defibrillation pulses. The lead 508C can include a proximal end that can be connected to the IMD 510 and a distal end that can be configured to be placed at a target location such as in a left ventricle (LV) 534 of the heart 505. The lead 508C may be implanted through the coronary sinus 533 and may be placed in a coronary vein over the LV such as to allow for delivery of one or more pacing pulses to the LV. The lead 508C can include an electrode 561 that can be located at a distal end of the lead 508C and another electrode 562 that can be located near the electrode 561. The electrodes 561 and 562 can be electrically connected to the IMD 510 such as via separate conductors in the lead 508C such as to allow for sensing of the LV electrogram and optionally allow delivery of one or more resynchronization pacing pulses from the LV.

The IMD 510 can include an electronic circuit that can sense a physiologic signal. The physiologic signal can include an electrogram or a signal representing mechanical function of the heart 505. The hermetically sealed can 512 may function as an electrode such as for sensing or pulse delivery. For example, an electrode from one or more of the leads 508A-C may be used together with the can 512 such as for unipolar sensing of an electrogram or for delivering one or more pacing pulses. A defibrillation electrode from the lead 508B may be used together with the can 512 such as for delivering one or more cardioversion/defibrillation pulses. In an example, the IMD 510 can sense impedance such as between electrodes located on one or more of the leads 508A-C or the can 512. The IMD 510 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 510 can be configured to inject current between an electrode on the RV lead 508B and the can 512, and to sense the resultant voltage between the same electrodes or between a different electrode on the RV lead 508B and the can 512. A physiologic signal can be sensed from one or more physiologic sensors that can be integrated within the IMD 510. The IMD 510 can also be configured to sense a physiologic signal from one or more external physiologic sensors or one or more external electrodes that can be coupled to the IMD 510. Examples of the physiologic signal can include one or more of heart rate, heart rate variability, intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are.

The CRM system 500 can include a patient chronic condition-based HF assessment circuit, such as illustrated in the commonly assigned Qi An et al., U.S. application Ser. No. 14/55,392, incorporated herein by reference in its entirety. The patient chronic condition-based HF assessment circuit can include a signal analyzer circuit and a risk stratification circuit. The signal analyzer circuit can receive patient chronic condition indicators and one or more physiologic signals from the patient, and select one or more patient-specific sensor signals or signal metrics from the physiologic signals. The signal analyzer circuit can receive the physiologic signals from the patient using the electrodes on one or more of the leads 508A-C, or physiologic sensors deployed on or within the patient and communicated with the IMD 510. The risk stratification circuit can generate a composite risk index indicative of the probability of the patient later developing an event of worsening of HF (e.g., an HF decompensation event) such as using the selected patient-specific sensor signals or signal metrics. The HF decompensation event can include one or more early precursors of an HF decompensation episode, or an event indicative of HF progression such as recovery or worsening of HF status.

The external system 520 can allow for programming of the IMD 510 and can receives information about one or more signals acquired by IMD 510, such as can be received via a communication link 503. The external system 520 can include a local external IMD programmer. The external system 520 can include a remote patient management system that can monitor patient status or adjust one or more therapies such as from a remote location.

The communication link 503 can include one or more of an inductive telemetry link, a radio-frequency telemetry link, or a telecommunication link, such as an internet connection. The communication link 503 can provide for data transmission between the IMD 510 and the external system 520. The transmitted data can include, for example, real-time physiologic data acquired by the IMD 510, physiologic data acquired by and stored in the IMD 510, therapy history data or data indicating IMD operational status stored in the IMD 510, one or more programming instructions to the IMD 510 such as to configure the IMD 510 to perform one or more actions that can include physiologic data acquisition such as using programmably specifiable sensing electrodes and configuration, device self-diagnostic test, or delivery of one or more therapies.

The patient chronic condition-based HF assessment circuit, or other assessment circuit, may be implemented at the external system 520, which can be configured to perform HF risk stratification such as using data extracted from the IMD 510 or data stored in a memory within the external system 520. Portions of patient chronic condition-based HF or other assessment circuit may be distributed between the IMD 510 and the external system 520.

Portions of the IMD 510 or the external system 520 can be implemented using hardware, software, or any combination of hardware and software. Portions of the IMD 510 or the external system 520 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 510, the CRM system 500 could include a subcutaneous medical device (e.g., subcutaneous ICD, subcutaneous diagnostic device), wearable medical devices (e.g., patch based sensing device), or other external medical devices.

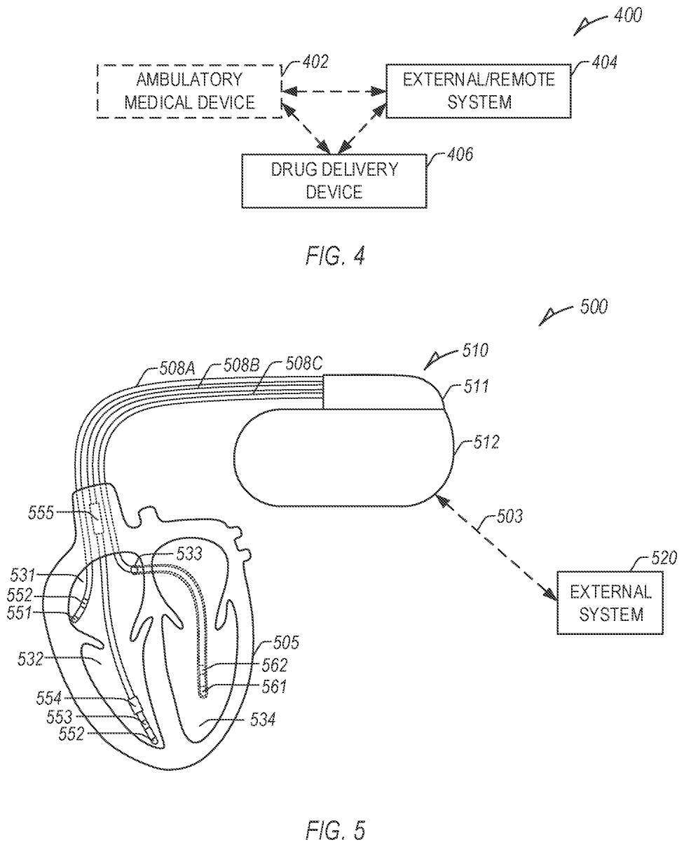

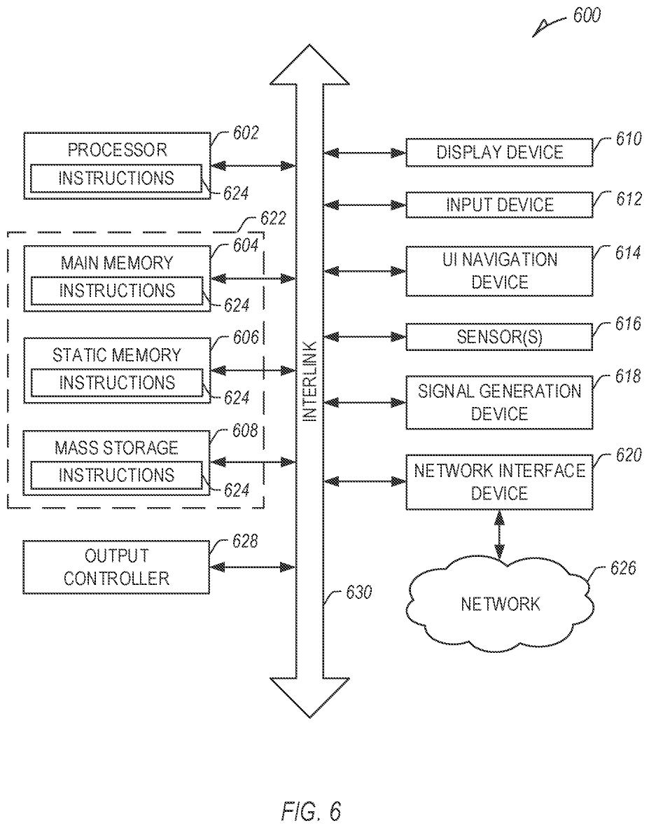

FIG. 6 illustrates a block diagram of an example machine 600 upon which any one or more of the techniques (e.g., methodologies) discussed herein may perform. Portions of this description may apply to the computing framework of one or more of the medical devices described herein, such as the IMD, the external programmer, etc.

Examples, as described herein, may include, or may operate by, logic or a number of components, or mechanisms in the machine 600. Circuitry (e.g., processing circuitry) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 600 may include a hardware processor 602 (e.g., a central processing unit (CPU), a graphics processing unit (GPU), a hardware processor core, or any combination thereof), a main memory 604, a static memory (e.g., memory or storage for firmware, microcode, a basic-input-output (BIOS), unified extensible firmware interface (UEFI), etc.) 606, and mass storage 608 (e.g., hard drive, tape drive, flash storage, or other block devices) some or all of which may communicate with each other via an interlink (e.g., bus) 630. The machine 600 may further include a display unit 610, an alphanumeric input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, optical media, magnetic media, and signals (e.g., radio frequency signals, other photon based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system comprising:
an ambulatory medical device configured to receive physiologic information from a patient; and
an assessment circuit configured to determine an indication of cardiotoxicity using the received physiologic information.

2. The system of claim 1, including a drug delivery system configured to control delivery of a cancer drug to the patient according to a delivery parameter,
wherein the assessment circuit is configured to determine an optimized delivery parameter using the determined indication of cardiotoxicity.

3. The system of claim 2, wherein the delivery parameter includes at least one of a dosage, a timing, or a drug.

4. The system of claim 1, wherein the assessment circuit is configured to provide an alert to a user using the determined indication of cardiotoxicity.

5. The system of claim 1, wherein the assessment circuit is configured to determine an adjusted therapy parameter using the determined indication of cardiotoxicity.

6. The system of claim 1, including a therapy circuit configured to control a therapy to the patient according to a therapy parameter,
wherein the assessment circuit is configured to adjust the therapy parameter using the determined indication of cardiotoxicity.

7. The system of claim 6, wherein the assessment circuit is configured to determine a cardiotoxicity index for the patient using the received physiologic information, to compare the determined cardiotoxicity index to a threshold, and, in response to the determined cardiotoxicity index exceeding the threshold, to adjust the therapy parameter using the determined cardiotoxicity index.

8. The system of claim 1, wherein the ambulatory medical device includes a heart sound sensor configured to receive heart sound information of the patient, and
wherein the assessment circuit is configured to determine the indication of cardiotoxicity using the received heart sound information.

9. The system of claim 8, wherein the assessment circuit is configured to determine an indication of reduced contractile function using the received heart sound information, and to determine the indication of cardiotoxicity using the determined indication of reduced contractile function.

10. The system of claim 9, wherein the assessment circuit is configured to detect a decrease in first heart sound (S1) amplitude using the received heart sound information, to determine the indication of reduced contractile function using the detected decrease in first heart sound (S1) amplitude, and to determine the indication of cardiotoxicity using the determined indication of reduced contractile function.

11. At least one machine-readable medium including instructions that, when performed by a medical device, cause the medical device to:
receive physiologic information from a patient; and
to determine an indication of cardiotoxicity using the received physiologic information.

12. The at least one machine-readable medium of claim 11, wherein the instructions, when performed by the medical device, cause the medical device to:
control delivery of a cancer drug to the patient according to a delivery parameter; and
determine an optimized delivery parameter using the determined indication of cardiotoxicity.

13. The at least one machine-readable medium of claim 12, wherein the delivery parameter includes at least one of a dosage, a timing, or a drug.

14. The at least one machine-readable medium of claim 11, wherein the instructions, when performed by the medical device, cause the medical device to:
provide an alert to a user using the determined indication of cardiotoxicity.

15. The at least one machine-readable medium of claim 11, wherein the instructions, when performed by the medical device, cause the medical device to:
determine an adjusted therapy parameter using the determined indication of cardiotoxicity.

16. A method comprising:
receiving physiologic information from a patient using an ambulatory medical device; and
determining, using an assessment circuit, an indication of cardiotoxicity using the received physiologic information.

17. The method of claim 16, including:
controlling, using the assessment circuit, delivery of a cancer drug to the patient according to a delivery parameter; and
determining, using the assessment circuit, an optimized delivery parameter using the determined indication of cardiotoxicity.

18. The method of claim 17, wherein the delivery parameter includes at least one of a dosage, a timing, or a drug.

19. The method of claim 16, including:
providing, using the assessment circuit, an alert to a user using the determined indication of cardiotoxicity.

20. The method of claim 16, including:
determining, using the assessment circuit, an adjusted therapy parameter using the determined indication of cardiotoxicity.
